(19) United States
(12) Patent Application Publication
(10) Pub. No.: US 2020/0360118 A1
(43) Pub. Date: Nov. 19, 2020

(54) SCANNABLE DENTAL SPATIAL ORIENTATION DEVICE AND RELATED SYSTEMS AND METHODS

(71) Applicant: Precision Dental Products, L.C., Draper, UT (US)

(72) Inventors: Juan Olivier, Draper, UT (US); Panaghiotis Bazos, Draper, UT (US)

(73) Assignee: Precision Dental Products, L.C.

(21) Appl. No.: 16/986,093

(22) Filed: Aug. 5, 2020

Related U.S. Application Data
(63) Continuation-in-part of application No. 16/286,344, filed on Feb. 26, 2019.
(60) Provisional application No. 62/635,387, filed on Feb. 26, 2018.

(51) Int. Cl. A61C 9/00; A61C 19/05; A61B 1/24

(52) U.S. Cl. CPC A61C 9/0053; A61B 2090/3937; A61B 1/24; A61C 19/05

(57) ABSTRACT

A scannable dental spatial orientation device can comprise a coupling feature operable to couple with a bite plate for supporting a physical dental record material. The scannable dental spatial orientation device can also comprise a body portion having first, second, and third planar faces oriented orthogonal to one another. In addition, the scannable dental spatial orientation device can comprise at least one joint configured for rotating the bite plate and the body portion relative to one another to align the first, second, and third planar faces parallel to a sagittal midline reference plane, a horizontal plane, and a frontal plane of a patient. A dental record optical scan system and a method for digitally recording a maxillary arch position are also disclosed.

### RELATED APPLICATION

This application is a continuation-in-part of U.S. application Ser. No. 16/286,344, filed Feb. 26, 2019, which claims the benefit of U.S. Provisional Application No. 62/635,387, filed Feb. 26, 2018 and each of which is incorporated herein by reference.

### BACKGROUND

The present invention relates generally to methods and apparatus for determining an occlusal or bite plane orientation and location. More particularly, the present invention relates to methods and apparatus for easily and accurately establishing and recording a patient's bite plane in relation to the relative horizontal and vertical alignment of the patient's face and head. The bite plane orientation may then be used in the fabrication of dental restorations and prosthetics.

While sitting or standing in a natural postural position, a patient's teeth typically should have an orientation that appears well centered and leveled in reference to the patient's facial features. Horizontal and vertical reference planes can be considered to create an attractive and natural looking set of teeth that also allow for proper mastication and occlusion. If a patient's face were perfectly symmetrical, the midline between the central incisors should be parallel and centered with the sagittal midline. Additionally, the patient's maxillary anterior incisal line, also known as the “bite plane” or “bite register”, should be parallel with the inter-pupillary eye line, a horizontal reference plane, and perpendicular with the sagittal mid-plane. However, very few individuals have perfect facial symmetry and dental orientation.

### SUMMARY

Often, bite impressions of the teeth necessary for dental restorations, prosthodontics, orthodontics or other procedures are taken without information regarding the orientation and location of the teeth or bite plane relative to the features of the face and head. Without the proper orientation, the resulting dental casts can lead to creation of dental restorations with improper occlusion of the patient's teeth and numerous aesthetic problems including an unnatural bite plane or a slanted maxillary midline and gum line. Some existing systems at least partially address these concerns but current systems continue to exhibit limitations.

Accordingly, a dental measurement device is disclosed that accurately captures the patient's occlusal bite plane and is easy to use. In one aspect, a scannable dental spatial orientation device is disclosed that can facilitate creation of a digital dental record that is related to position information of the maxillary arch relative to the sagittal midline reference plane, the horizontal plane, and the frontal plane of the patient. The digital dental record can be used to create a maxillary model. The scannable dental spatial orientation device can comprise a coupling feature operable to couple with a bite plate for supporting a physical dental record material. The scannable dental spatial orientation device can also comprise a body portion having first, second, and third planar faces oriented orthogonal to one another. In addition, the scannable dental spatial orientation device can comprise at least one joint configured for rotating the bite plate and the body portion relative to one another to align the first, second, and third planar faces parallel to a sagittal midline reference plane, a horizontal plane, and a frontal plane of a patient.

A dental record optical scan system is also disclosed. The dental record optical scan system can comprise a physical dental record of a maxillary arch of a patient. The dental record optical scan system can also comprise a scannable dental spatial orientation device associated with the physical dental record. The scannable dental spatial orientation device can include a body portion having first, second, and third planar faces oriented orthogonal to one another and representing planes aligned parallel to a sagittal midline reference plane, a horizontal plane, and a frontal plane of the patient relative to the physical dental record. The dental record optical scan system can further comprise a support structure operable to support the physical dental record and the scannable dental spatial orientation device for optical scanning. Additionally, the dental record optical scan system can comprise an optical scanner operable to scan the physical dental record and the scannable dental spatial orientation device with an optical sensor to create a digital dental record and a digital representation of the first, second, and third planar faces. The digital dental record can be established for a position of the maxillary arch relative to the sagittal midline reference plane, the horizontal plane, and the frontal plane of the patient.

In addition, a method for digitally recording a maxillary arch position is disclosed. The method can comprise obtaining a physical dental record of a maxillary arch of a patient and a scannable dental spatial orientation device associated with the physical dental record, the scannable dental spatial orientation device including a body portion having first, second, and third planar faces oriented orthogonal to one another and representing planes aligned parallel to a sagittal midline reference plane, a horizontal plane, and a frontal plane of the patient relative to the physical dental record. The method can also comprise coupling the scannable dental spatial orientation device to a support structure operable to support the physical dental record and the scannable dental spatial orientation device for optical scanning. In addition, the method can comprise optically scanning the physical dental record and the scannable dental spatial orientation device to create a digital dental record and a digital representation of the first, second, and third planar faces, wherein the digital dental record is established for a position of the maxillary arch relative to the sagittal midline reference plane, the horizontal plane, and the frontal plane of the patient.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A illustrates a front-top perspective view of a dental measurement device in accordance with an example of the present disclosure.

FIG. 1B illustrates a rear-top perspective view of the dental measurement device of FIG. 1A.

FIG. 1C illustrates a rear-bottom perspective view of the dental measurement device of FIG. 1A.

FIG. 1D illustrates a top-front exploded perspective view of the dental measurement device of FIG. 1A.

FIGS. 2A-2D illustrate the dental measurement device of FIG. 1A used to establish the orientation of a dental bite plane in relation to the facial planes of symmetry and orientation of a patient, in accordance with an example of the present disclosure.

FIG. 3 illustrates a bite plate or tray in accordance with an example of the present disclosure.

FIGS. 4A and 4B illustrate exploded perspective views of the scannable spatial orientation cube (XYZ virtual orientation) and the arm of the dental measurement device of FIG. 1A.

FIG. 5 illustrates a scannable dental spatial orientation device in accordance with an example of the present disclosure.

FIG. 6 illustrates a bite plate or tray including an optical sensor recognition feature in accordance with an example of the present disclosure.

FIGS. 7A and 7B illustrate a dental record optical scan system in accordance with an example of the present disclosure.

FIG. 8 illustrates a support structure operable to support a physical dental record and a scannable dental spatial orientation device for optical scanning, in accordance with an example of the present disclosure.

FIG. 9A illustrates a front view of a dental measurement device in accordance with another example of the present disclosure.

FIG. 9B illustrates a side view of the dental measurement device of FIG. 9A.

FIG. 10A illustrates a nose extension arm in accordance with an example of the present disclosure.

FIG. 10B illustrates a removably attachable level coupled to the nose extension arm of FIG. 10A in accordance with an example of the present disclosure.

FIG. 11 illustrates a first Fox plane frame in accordance with an example of the present disclosure.

FIG. 12 illustrates a second Fox plane frame in accordance with an example of the present disclosure.

FIG. 13 illustrates a pupillary bar in accordance with an example of the present disclosure.

FIG. 14 illustrates a dental prosthetic manufacturing system mounted on an articulator in accordance with an example of the present disclosure.

FIG. 15A illustrates a side view of a stand for supporting a bite plate on an articulator, in accordance with an example of the present disclosure.

FIG. 15B illustrates a rear-top perspective view of the stand of FIG. 15A.

FIG. 15C illustrates a top view of the stand of FIG. 15A with a transfer plate omitted.

FIG. 15D illustrates a top view of a base of the stand of FIG. 15A.

FIG. 16 illustrates a slider mount for coupling a dental measurement device to an articulator, in accordance with an example of the present disclosure.

FIG. 17 illustrates a dental prosthetic manufacturing system mounted on an articulator in accordance with an example of the present disclosure.

FIG. 18 illustrates the dental prosthetic manufacturing system of FIG. 17 isolated from the articulator.

FIG. 19 illustrates an exploded view of certain components of the dental prosthetic manufacturing system of FIG. 17.

FIGS. 20A-20C illustrate a reference tool for modeling a dental prosthetic in accordance with an example of the present disclosure.

FIG. 21 illustrates a reference tool for modeling a dental prosthetic in accordance with an example of the present disclosure.

FIG. 22 illustrates a reference tool for modeling a dental prosthetic in accordance with an example of the present disclosure.

FIG. 23 illustrates a stylus that facilitates construction of a maxillary model for use on an articulator, in accordance with an example of the present disclosure.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a bar” includes reference to one or more of such components and reference to “aligning” refers to one or more of such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Dental Measurement Device and Prosthetic Manufacturing System

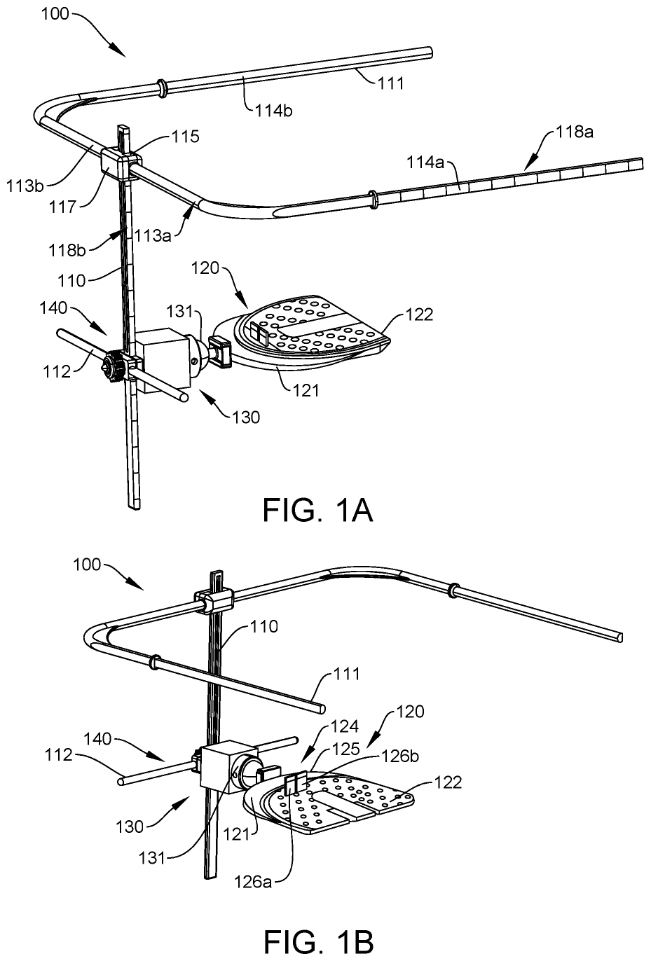

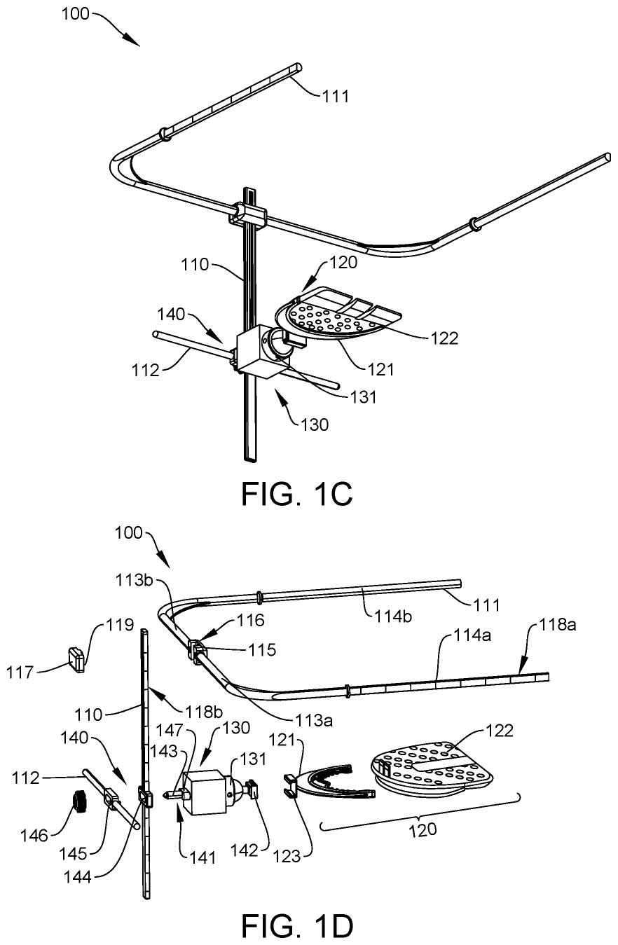

With reference to FIGS. 1A-1D, a dental measurement device 100 is illustrated in accordance with an example of the present disclosure. The present disclosure includes methods of establishing occlusal bite planes in relation to anatomical planes of symmetry and orientation. The sagittal midline reference plane or line lies in the anterior-posterior direction. The sagittal midline reference plane bisects a patient's body into theoretically symmetrical right and left halves. The horizontal reference plane is a general reference plane established by having the patient sit upright in a natural fashion and then envisioning a plane that generally describes the apparent level horizontal plane of the patient's head. The horizontal reference plane is perpendicular to the sagittal midline reference plane. The frontal reference plane is another general plane established by having the patient sit straight upright in a natural fashion and then envisioning a plane running across the face that generally describes the overall plane of the patient's face. The frontal reference plane is generally perpendicular to the sagittal midline reference plane and generally perpendicular to the horizontal base plane. It will be appreciated that the exact angle of each of these planes and lines and their relationship to one another, will vary from individual to individual. Knowing the orientation of the bite plane or bite register relative to the patient's face and head helps to create dental restorations and prostheses that are more functional, comfortable and attractive.

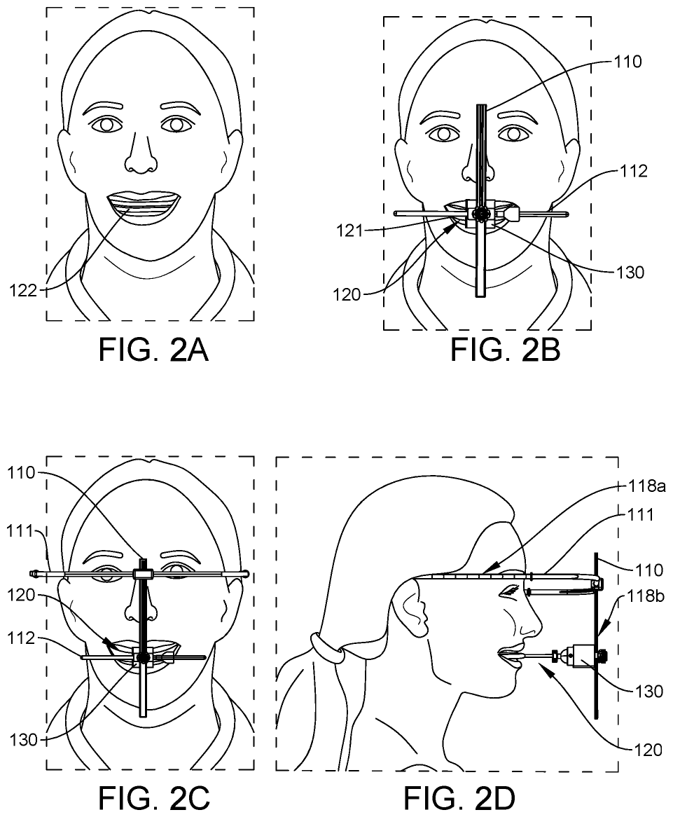

The dental measurement device 100 can include orientation components to align with and/or define the various anatomical planes of symmetry and orientation. For example, the dental measurement device 100 can include a vertical or midline orientation rod 110 to align with a sagittal midline reference plane of a patient. The dental measurement device 100 can also include a baseline orientation frame 111 coupleable to the vertical orientation rod 110 to align with one or more landmarks, e.g. the corner of the ear (right otobasion superius) and corner of the eye (right exocanthion) of the patient to define a horizontal base plane of the patient. Such eye and ear features of a patient are discussed in more detail below with respect to FIGS. 2C and 2D. In some embodiments, the dental measurement device 100 can include a horizontal orientation rod 112 coupleable to the vertical orientation rod to align with the horizontal reference plane of the patient.

As shown in FIGS. 1A and 1D, the baseline orientation frame 111 can include left and right lateral extension bars 113a, 113b configured to extend at least to the width of the patient's head. The baseline orientation frame 111 can also include left and right side bars 114a, 114b configured to extend rearward from the respective left and right lateral extension bars 113a, 113b at least to the patient's ears. The baseline orientation frame 111 can include a center bracket portion 115 with an opening 116 (FIG. 1D) configured to fit about the vertical orientation rod 110. The baseline orientation frame 111 can include an attachment clip 117 operable to couple with the center bracket portion 115 to secure the baseline orientation frame 111 to the vertical orientation rod 110. The attachment clip 117 can include friction enhancing features 119 (e.g., teeth, ridges, etc.) to prevent slippage of the baseline orientation frame 111 relative to the vertical orientation rod 110. The attachment clip 117 can be coupled to the center bracket portion 115 in any suitable manner, such as by magnets. In one aspect, the baseline orientation frame 111 can include reference markings 118a, which can assist a technician in properly positioning a dental record recorded using the device 100 in an articular, as described in more detail below. Similarly, the vertical orientation rod 110 can include reference markings 118b for the same reason. In some embodiments, the vertical orientation rod 110 and the baseline orientation frame 111 an be made of metal (e.g., a ferrimagnetic and/or a ferromagnetic material), which can provide durability for reuse of the components and enable the use of magnets to couple the components to one another.

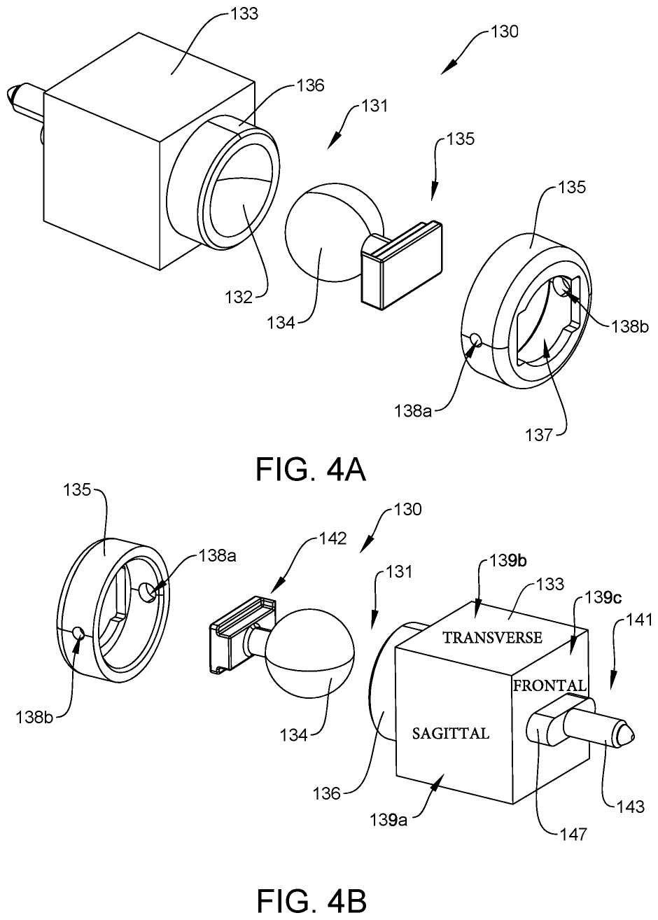

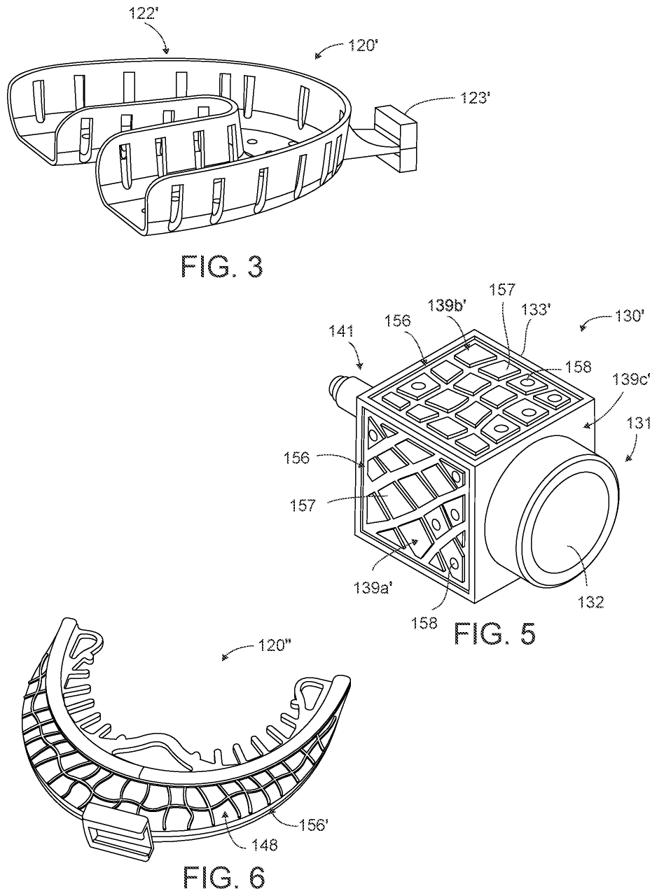

The dental measurement device 100 can further include a bite plate or tray 120 configured to be held in the mouth of the patient and support impression or cast material (not shown) used to make a maxillary cast of the patient's teeth. A bite plate or tray as disclosed herein can have any suitable configuration for supporting impression or cast material (e.g., dental record material), such as a planar configuration as illustrated in the bite plate or tray 120 of FIGS. 1A-1D or a channel configuration as illustrated in another example of a bite plate or tray 120′ in FIG. 3. In addition, the dental measurement device 100 includes an extension arm or holder 130 (shown isolated in FIGS. 4A and 4B) for coupling the bite plate 120 to the vertical orientation rod 110. The present disclosure can also utilize aspects of devices and methods for establishing the orientation of a dental bite plane that are disclosed in U.S. Pat. Nos. 7,364,429 and 7,399,182, the disclosure of each of these references is incorporated in its entirety herein.

The bite plate 120 may be sized and configured to fit in the patient's mouth and may be held in place by the upper and lower teeth or gums of the patient. The bite plate 120 may be of various sizes to accommodate different mouth sizes in small children, adolescents, and adults. In some embodiments, the bite plate 120 can comprise an arch portion 121 (sometimes referred to as a “bite fork”) or generally U-shape sized to fit in the patient's mouth, which may be held in place by the patient's teeth or gums. An impression of the patient's teeth can be made in the bite plate or bite impression material, which can record the patient's incisal edge position. In one aspect, the bite plate 120 can comprise an occlusal plate or tray 122 fixedly coupleable to the arch portion, which may extend across the patient's tongue. Alternatively, as illustrated with the bite plate or tray 120′ in FIG. 3, a bite plate or tray as disclosed herein can have an arch portion and/or an occlusal plate or tray portion 122′ integrally formed in a monolithic structure.

The bite plate 120 may be attached to the orientation components (e.g., the vertical orientation rod 110) via a connection system 140, which can include the extension arm 130 and other coupling components described herein. The connection system 140 may be configured to allow alignment of the vertical orientation rod 110, the baseline orientation frame 111, and the horizontal orientation rod 112 with the vertical, horizontal, and/or frontal planes of the patient's face and head, as applicable. Once an orientation component is correctly adjusted, the connection system 140 may be locked into place to preserve the orientation of the patient's bite plane relative to the facial reference planes.

In one aspect, the bite plate 120 can be movable (e.g., translatable and/or rotatable) relative to the vertical orientation rod 110. For example, the bite plate 120 can be rotatable relative to the vertical orientation rod 110 to facilitate recording a frontal plane of the patient. In one embodiment, the extension arm 130 can comprise a main body portion 133 coupleable to the vertical orientation rod 110 in a fixed relationship. The extension arm 130 can also comprise at least one joint 131 configured for rotating the bite plate 120 relative to the main body portion 133. Thus, the at least one joint 131 can rotate the bite plate 120 relative to the vertical orientation rod 110. In the illustrated embodiment, the at least one joint 131 is configured to rotate the bite plate 120 relative to the main body portion 133 and the vertical orientation rod 110 in up to three rotational degrees of freedom, i.e., parallel to the sagittal midline reference plane, the horizontal plane, and/or the frontal plane. This is achieved, in the illustrated embodiment, by a ball and socket joint. In particular, a socket 132 is associated with the main body portion 133 of the extension arm 130.

The main body portion 133 can be associated with a coupling feature 141 configured to couple the extension arm 130 to the vertical orientation rod 110. The socket 132 is configured to receive and interface with a ball 134, which is associated with a coupling feature 142 configured to couple with a mating coupling feature 123 of the bite plate 120 (or a mating coupling feature 123′ of the bite plate 120′ in FIG. 3) to couple the extension arm 130 to the bite plate 120 (or the bite plate 120′). The ball 134 is secured to the socket 132 by a securing ring 135, which can couple with an outer ring 136 about the socket 132. The securing ring 135 can include an opening sized and shaped to fit over the coupling feature 135. In the illustrated embodiment, the coupling feature 135 has a rectangular outer shape. The securing ring 135 has an opening 137 configured to accommodate passage of the coupling feature 135 through the opening 137 while also being configured to capture the ball at least partially within the socket 132. The securing ring 135 can also include one or more injection openings 138a, 138b to facilitate application of an adhesive through the injection openings 138a, 138b to fix the ball 134 and socket 132 relative to one another and prevent relative movement to preserve an orientation of the bite plate 120 relative to the orientation components (e.g., the vertical orientation bar 110). In one aspect, one or more of the injection openings 138a, 138b can be threaded to receive a set screw (not shown) to at least temporarily hold the joint 131 in a fixed position during adjustments to properly orient the baseline orientation frame 111, as discussed in more detail below.

In one aspect, the only movable components may be the bite plate 120 rotatable relative to the vertical orientation rod (e.g., via the ball and socket joint 131 of the extension arm 130), and the baseline orientation frame 111 translatable relative to the vertical orientation rod 110. All other positional or angular relationships may be fixed or maintained constant.

Although only a single joint 131 is illustrated in this embodiment, it should be recognized that any suitable arrangement of joints and joint configurations can be utilized to achieve a desired rotation of the bite plate 120 relative to the vertical orientation rod 110. In addition, in some embodiments, a ball and socket joint can be configured such that the ball 134 is associated with the main body portion 133 and the socket 132 is associated with the coupling feature 142. Furthermore, in some embodiments, the extension arm 130 can be configured such that the coupling feature 142 is associated with the main body portion 133.

It should also be recognized that the coupling features described herein can have any suitable design or configuration. For example, the coupling features 142, 123 are configured for a lateral sliding engagement and can rely on friction to maintain the connection. The coupling feature 141 can include a threaded rod 143 configured to extend through openings 144, 145 in the vertical and horizontal orientation rods, respectively, and be secured by a nut 146 (see FIG. 1D). In one aspect, the openings 144, 145 can be elongated laterally parallel to the horizontal plane to enable lateral translation of the bite plate 120 relative to the vertical orientation rod 110 in a direction parallel to the horizontal plane. In another embodiment, the coupling feature 141 can include a shoulder 147 configured to fit within at least one of the openings 144, 145 and occupy enough of the openings to prevent relative lateral translational movement of the bite plate 120 and the vertical orientation rod 110 in a direction parallel to the horizontal plane. The shoulder 147 can also be configured to prevent relative rotational movement between the bite plate 120 and the vertical orientation rod 110. In this case, the bite plate 120 can be configured to include a center indicator 124 for facial midline recording (e.g., a slot or opening 125 between center tabs 126a, 126b (see FIG. 1B), a recess, a protrusion, a block, a mark, etc.), which can represent the location of a future dental midline if coincident with the facial midline when the record is being utilized with an articulator to construct a dental prosthetic, as discussed below.

The main body portion 133 can have any suitable shape or configuration. In the illustrated embodiment, the main body portion 133 comprises a rectangular cuboid (e.g., a “cube”) configuration. In one aspect, one or more orthogonal planar faces 139a-c of the “cube” (e.g., sagittal, transverse, and frontal faces of the cube as shown in FIG. 4B) can be oriented parallel to orthogonal facial reference planes of the patient (e.g., midline, horizontal, and frontal reference planes), as described herein. For example, the main body portion 133 can be fixedly coupled (i.e., constrained with no relative rotation and translation) to the vertical orientation bar 110 in a manner that aligns the sagittal, transverse, and frontal faces 139a-c or surfaces of the cube parallel with the respective midline, horizontal, and frontal reference planes of the patient established by the dental measurement device 100, as described herein (see, e.g., discussion with regard to FIGS. 2A-2D). The at least one joint 131 can be configured to rotate the bite plate 120 and the body portion 133 relative to one another to align the planar faces 139a-c parallel to a sagittal midline reference plane, a horizontal plane, and a frontal plane of a patient. As described above, the at least one joint 131 can be configured for rotating the bite plate 120 relative to the body portion 120 in a direction parallel to at least one of the planar faces 139a-c of the cube to align the at least one of the planar faces 139a-c parallel to a sagittal midline reference plane, a horizontal plane, and/or a frontal plane of a patient. The coupling feature 141 can be operable to couple with the vertical orientation rod 110 to facilitate aligning at least one of the planar faces 139a-c parallel to the sagittal midline reference plane, the horizontal plane, and/or the frontal plane of the patient.

The dental measurement device 100 can be used to establish the orientation of a dental bite plane in relation to the facial planes of symmetry and orientation of a patient, as shown in FIGS. 2A-2D. For example, after placing bite material on the occlusal plate 122, the occlusal plate 122 can be placed in the patient's mouth engaging the teeth, as shown in FIG. 2A. The arch portion 121 can then be connected to the occlusal plate 122, the extension arm 130 connected to the arch portion 121, and the vertical orientation rod 110 connected to the extension arm 130, as shown in FIG. 2B, and the baseline orientation frame 111 can be connected to the vertical orientation rod 110, as shown in FIG. 2C. The horizontal orientation rod 112 can optionally be connected to the vertical orientation rod 110, as shown in FIG. 2B. These connections can be done in any suitable order with the occlusal plate 122 located inside or outside of the patient's mouth.

In one aspect, a line from an eye feature or landmark, such as the corner of the patient's eye (i.e., the exocanthion), to an ear feature or landmark, such as the top of the ear (i.e., the otobasion superius), can serve as a “baseline” or foundational reference for setting up the dental measurement device 100 relative to the patient to determine a position and orientation of the maxillary arch that can be translated to an articulator. Accordingly, the baseline orientation frame 111 can be positioned along the vertical orientation rod 110 and the vertical orientation rod 110 can be rotated relative to the bite plate 120 such that the baseline orientation frame 111 aligns with eye and ear features or landmarks of the patient, specifically the corner of the eye (i.e., the exocanthion) and the top of the ear (i.e., the otobasion superius), as shown in FIG. 2D. Alignment can be viewed from the right and/or left sides of the patient. FIG. 2D also illustrates that the extension arm 130 can have a length sufficient to position the vertical orientation rod 110 beyond the tip of the nose of the patient.

With the baseline orientation frame 111 properly positioned, the joint 131 can be locked or fixed in position by applying an adhesive to the joint 131, such as by injecting adhesive into the injection openings 138a, 138b (see FIGS. 4A and 4B). If a set screw was utilized in one or more of the injection openings 138a, 138b to assist in securing the joint 130 during adjustment, the set screw can be removed, if desired. Locking or fixing the joint in a permanent position can capture and preserve the orientation of the bite plate 120 relative to horizontal, midline, and frontal reference planes. A photograph from a side (e.g., the right side) as in FIG. 2D can preserve a visual record of the position of the baseline orientation frame 111 relative to the patient's temporal mandibular joint and the position of the baseline orientation frame 111 relative to the vertical orientation rod 110, as indicated by the reference markings 118a, 118b, respectively. Thus, the dental measurement device 100 can enable a maxillary imprint or cast to be positioned on an articulator in very close to the same position and orientation relative to the condylar of the articulator as the maxillary arch is positioned and oriented relative to the temporal mandibular joint of the patient. The bite plate 120 and the extension arm 130 along with a photograph showing a side view of the dental measurement device 100 can be provided to a laboratory for use with an articulator. All other components of the dental measurement device 100 can be reused to record the bite plane of another patient.

As most individuals vary from having perfectly perpendicular and perfectly proportioned features (for example the lips may be slightly crooked on the patient's face), the recordation of facial and dental features by the present technology accommodates variance in patient symmetry and anatomy and allows for the creation of dental prosthetics that appear more attractive on the patient.

In some examples, an extension arm can be configured as a scannable dental spatial orientation device. Such an extension arm 130′ in accordance with one example of the present disclosure is illustrated in FIG. 5. As with the extension arm 130 disclosed herein, the extension arm 130′ can include a body portion 133′ and at least one joint 131 configured for rotating the bite plate 120 and the body portion 133′ relative to one another. The joint 131 may be configured similar to the corresponding joint of the extension arm 130 discussed above and therefore can comprise a ball and socket joint. In the example extension arm 130′ illustrated in FIG. 5, the socket 132 is illustrated but the ball 134 and associated coupling feature 142 for coupling with the bite plate 120 have been omitted from the figure. As with the extension arm 130 discussed above, the extension arm 130′ can also include a coupling feature 141 configured to couple the extension arm 130′ to the vertical orientation rod 110.

In further similarity to the extension arm 130, the extension arm 130′ can also include planar faces 139a′-c′ oriented orthogonal to one another, and the at least one joint 131 can be configured to rotate the bite plate and the body portion 133 relative to one another to align the planar faces 139a′-c′ parallel to a sagittal midline reference plane, a horizontal plane, and/or a frontal plane of a patient. As illustrated in FIG. 5, the extension arm 130′ can have a rectangular cuboid configuration (e.g., a “cube” configuration). In one aspect, one or more planar faces 139a′-c′ of the cube can be oriented parallel to one or more facial reference planes of the patient, as described above. In the case of the extension arm 130′ configured as a scannable dental spatial orientation device, the one or more planar faces 139a′-c′ of the extension arm 130′ can be utilized to define orthogonal reference planes or axes (e.g., X, Y, and Z axes) for a dental record (e.g., a digital version, model, or representation of the physical dental record supported by the bite plate 120) using suitable a digitization device (e.g., a scanner, such as a three-dimensional optical scanner, commonly used in dental offices and laboratories for high precision dental prosthetic manufacturing).

Featureless, flat and/or smooth surfaces may confuse an optical sensor and associated software and therefore may not be recognized by an optical sensor and related software. Therefore, in one aspect, at least a portion of one or more planar faces 139a′-c′ of the extension arm 130′ can include an optical sensor recognition feature 156 operable to facilitate recognition by an optical sensor (e.g., a camera, a scanner, etc.) for digitally capturing or preserving a representation of the dental record, as described in more detail below. The one or more (e.g., three) planar faces 139a′-c′ can define the X, Y, and/or Z three-dimensional scannable orientation (e.g., alignment) of the cranio-maxillary complex. At least one of the planar faces 139a′-c′ can comprise an optical sensor recognition feature 156 configured to facilitate recognition of the at least one of the planar faces 139a′-c′ by an optical sensor. The optical sensor recognition feature 156 can be or include any suitable surface treatment or feature (e.g., a two-dimensional feature and/or a three-dimensional feature), such as a matte finish, a surface irregularity (e.g., a hole), a texture, a shape (e.g., a geometric shape and/or a freeform shape), a pattern (e.g., a reticulate pattern, a parametric pattern, and/or a fractal pattern), an alphanumeric character, and/or indicia (e.g., a company name, a logo, and/or a trademark). In one example, optical sensor recognition features 156 can include a reticulation/parametric pattern 157 on one or more sides or faces 139a′-c′ of a cube to facilitate seamless scanning, with one or more holes 158 on the faces 139a′-c′ that extend deeper into the cube than the reticulation/parametric pattern 157 to facilitate digital meshing.

An optical sensor recognition feature as disclosed herein can be included with any suitable part or component of a measurement device. For example, as shown in FIG. 6, a bite plate 120″ can include an optical sensor recognition feature 156′, as described above, which is configured to facilitate recognition of a planar face 148 of the bite plate 120″ by an optical sensor.

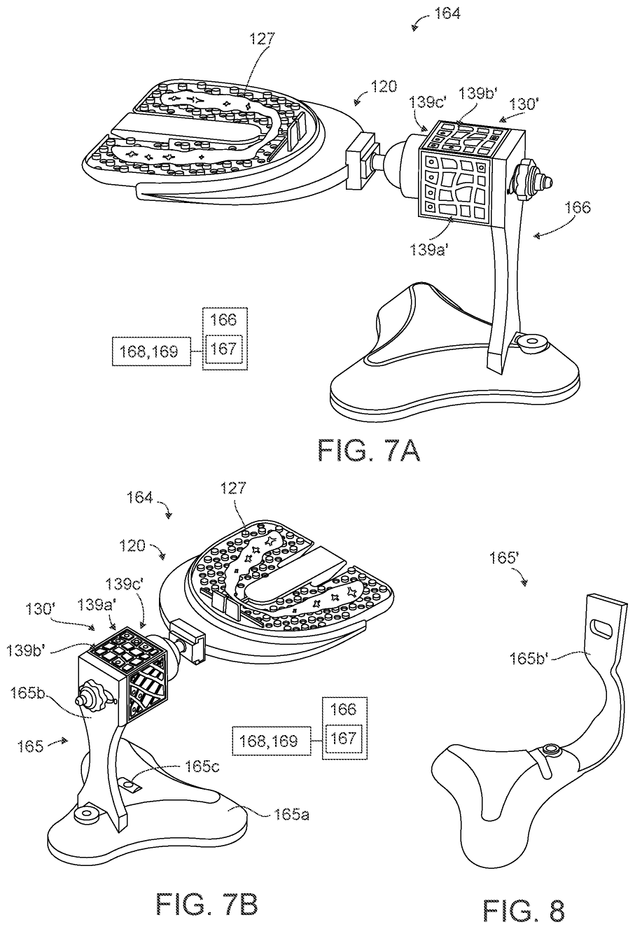

FIGS. 7A and 7B illustrate a dental record optical scan system 164 in accordance with an example of the present disclosure. The system can include a physical dental record 127 of a maxillary arch of a patient. The physical dental record can be supported by the bite plate 120 (or the bite plate 120′). The system 164 can also include a scannable dental spatial orientation device 130′ associated with the physical dental record 127 in the manner disclosed herein, such as by a coupling with the bite plate 120. The system 164 can further include a support structure 165 (e.g., a cube holder) operable to support the physical dental record 127 and the scannable dental spatial orientation device 130′ for optical scanning. Additionally, the system can include an optical scanner 166 operable to scan the physical dental record 127 and the scannable dental spatial orientation device 130′ with an optical sensor 167 to create a digital dental record 168 and a digital representation 169 of the planar faces 139a′-c′. The digital dental record 168 can be established for a position of the maxillary arch relative to the sagittal midline reference plane, the horizontal plane, and the frontal plane of the patient.

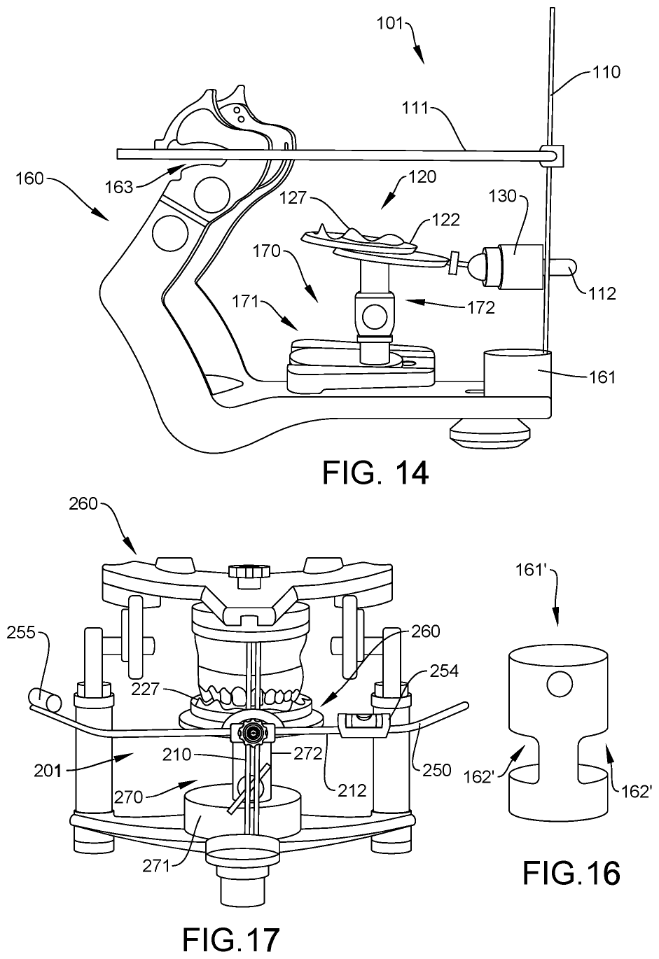

The optical scanner 166 can be or include any suitable type of optical scanner known in the art, such as an intraoral scanner and/or a desktop dental scanner. The digital dental record 168 and the digital representation 169 of the planar faces 139a′-c′ can be created by computer software executed on a computer associated with the optical scanner 166, as commonly known in the art. In addition, the digital dental record 168 and the digital representation 169 of the planar faces 139a′-c′ can be stored in computer memory, as known in the art, and therefore may be copied, transferred, and utilized by suitable software (e.g., dental CAD/CAM software) for any purpose, such as to create a physical facsimile of the original physical dental record 127 and/or a maxillary model that will be mounted on an articulator. Thus, the digital dental record 168 and the digital representation 169 of the planar faces 139a′-c′ can be used by a technician, in addition to or instead of, the physical dental record 127 and associated physical reference features (e.g., as described with reference to FIG. 14 below) to create a maxillary model.

The support structure 165 can include a base portion 165a and a support arm 165b extending vertically from the base portion 165a. The support arm 165b can be configured to interface and couple with the scannable dental spatial orientation device 130′, such as utilizing the coupling feature 141 described above (e.g., a threaded fastener such as the threaded rod 143 and nut 146). In addition, the support arm 165b can include an opening configured to receive the shoulder 147 of the scannable dental spatial orientation device 130′ to prevent relative rotational and translational movement between the scannable dental spatial orientation device 130′ and the support arm 165b.

The base portion 165a can be operable to support the physical dental record 127 and the scannable dental spatial orientation device 130′ relative to a support surface for optical scanning. The base portion 165a can have any suitable shape or configuration, such as a triangular configuration (as illustrated), a rectangular configuration, a circular configuration, etc. The support arm 165b can have any suitable configuration, such as the straight support arm 165b configuration as shown in FIGS. 7A and 7B, or a curved support arm 165b′ configuration as shown in FIG. 8. The straight support arm 165b configuration can provide access and clearance for an intraoral scanning wand typically used by clinicians. The curved support arm 165b′ configuration may be suitable for use with a desktop dental scanner typically used by technicians. In one aspect, the base portion 165a can include a level 165c to facilitate orienting one of the first, second, or third planar faces in a horizontal plane. For example, the level 165c (e.g., a bubble level) can be located in the center of the base portion 165a to ensure that the top planar face 139b′ of the scannable dental spatial orientation device 130′ is in a horizontal orientation. The support structure 165 and its various components can be made of any suitable material, such as aluminum, steel, polymer, composite, etc.

In order to digitally record a maxillary arch position, the scannable dental spatial orientation device 130′ and the bite plate 120 (or the bite plate 120′), with bite registration material, can first record the position of the patient's maxillary arch as described herein, and create a physical dental record 127. The scannable dental spatial orientation device 130′ and the bite plate 120 (or the bite plate 120′) can then be coupled to the support structure 165, such as by inserting a threaded rod through an opening in the support arm 165b and securing the scannable dental spatial orientation device 130′ to the support arm 165b with a nut threaded onto the threaded rod. With the scannable dental spatial orientation device 130′ attached to, and supported by, the support structure 165, a clinician or technician can then use an optical scanner 166 (e.g., an intraoral scanner or a desktop dental scanner) to scan the physical dental record 127 (e.g., the bite material that is attached to the bite plate 120 (or the bite plate 120′)) and the scannable dental spatial orientation device 130′ to create a digital dental record 168 and a digital representation 169 of the planar faces 139a-c. The digital dental record 168 can thereby be established for a position of the maxillary arch relative to the sagittal midline reference plane, the horizontal plane, and the frontal plane of the patient.

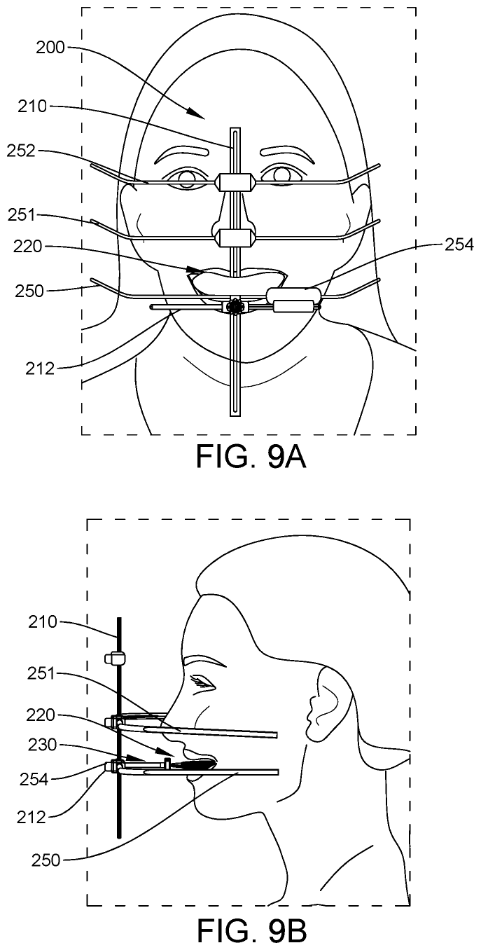

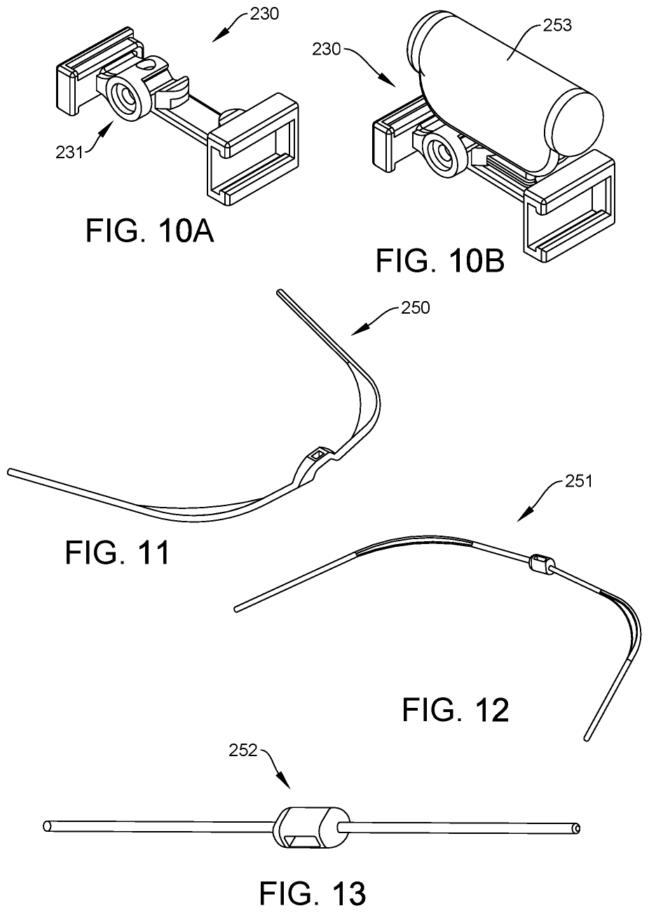

FIGS. 9A and 9B illustrate a dental measurement device 200 in accordance with another example of the present disclosure. The dental measurement device 200 is similar to the dental measurement device 100 in many respects. For example, the dental measurement device 200 can comprise a vertical or midline orientation rod 210 to align with a sagittal midline reference plane of a patient, a horizontal orientation rod 212 coupleable to the vertical orientation rod 210 to align with the horizontal reference plane of the patient, a bite plate 220 configured to be held in the mouth of the patient and support impression or cast material used to make a maxillary cast of the patient's teeth, and a nose extension arm 230 (shown isolated in FIG. 10A) for coupling the bite plate 220 to the vertical orientation rod 210. The dental measurement device 200 can also include a first Fox plane frame 250 (shown isolated in FIG. 11), a second Fox plane frame 251 (shown isolated in FIG. 12), and/or a pupillary rod 252 (shown isolated in FIG. 13) to enhance the ability to accurately establish the orientation of a dental bite plane. In some embodiments, the first Fox plane frame 250 can be coupled to the vertical orientation rod 210 proximate the horizontal orientation rod 212. The second Fox plane frame 251 can be movably coupled to the vertical orientation rod 210 above the first Fox plane 250. The pupillary rod 252 can also be movably coupled to the vertical orientation rod 210.

In one aspect, the bite plate 220 can be movable (e.g., translatable and/or rotatable) relative to the vertical orientation rod 210. For example, the bite plate 220 can be rotatable relative to the vertical orientation rod 210 to facilitate recording a frontal plane of the patient. In one embodiment, the nose extension arm 230 can comprise at least one joint 231 (FIG. 10A) configured for rotating the bite plate 220 relative to the vertical orientation rod 210. In the illustrated embodiment, the at least one joint 231 is configured to rotate the bite plate 220 relative to the vertical orientation rod 210 in one rotational degree of freedom, i.e., parallel to the sagittal midline reference plane. This is achieved, in the illustrated embodiment, by a hinge joint.

In some embodiments, the bite plate 220 (i.e., the occlusal plate) can have a ball 228 or a socket (see FIG. 19) on a bottom side of the occlusal plate to facilitate rotatably coupling the bite plate 220 to an articulator used in manufacturing a dental prosthetic, as described in more detail below. In some cases, the ball 228 or socket joint member can be integrally formed into the bottom side. Alternatively, the joint member can be removably coupleable to the bottom side.

The dental measurement device 200 can be used to establish the orientation of a dental bite plane in relation to the facial planes of symmetry of a patient by placing the bite plate 220 into the patient's mouth while engaging the teeth (with the bite plate 220, or by placing a separate bite impression material into the bite plate 220), such that a connection system (e.g., structure including the nose extension arm 230) extends from the bite plate 220 and out of the mouth of the patient. The vertical orientation rod 210 and the horizontal orientation rod 212 may then be connected to the nose extension arm 230, which can also be connected to the bite plate 220 (e.g., via connection plates fitting within corresponding connection housings or receptacles). The nose extension arm 230 can have a length sufficient to position the vertical and horizontal orientation rods 210, 212 beyond a tip of a nose of the patient.

The vertical orientation rod 210 may be adjusted by rotation and/or lateral motion until it is substantially parallel with the sagittal midline of the patient's head. Upon adjustment, the vertical orientation rod 210 may be dead center on the contact point of the middle of the center incisors. The vertical orientation rod 210 records the sagittal midline reference plane or line. In some embodiments, the vertical orientation rod 210 may also be adjusted to be substantially parallel to the frontal plane of the patient's head. For example, the nose extension arm 230 can include the joint 231 configured for rotating the bite plate 220 and the vertical orientation rod 210 relative to one another to facilitate recording a frontal or facial plane of the patient. The nose extension arm 230 can be configured to couple with a level 253 (see FIG. 10B) (e.g., a bubble level), which can be used to adjust the vertical orientation rod such that it is vertical in a direction parallel to the midline plane.

The horizontal orientation rod 212 can be adjusted by rotation until it is substantially parallel with a horizontal reference plane, which can be facilitated by using a detachable level 254 (e.g., a bubble level). The horizontal orientation rod 212 records the horizontal reference plane or line. The pupillary rod 252 can be aligned with pupils of the patient to provide a physical representation of the interpupillary line, which may be in a horizontal reference plane in most patients and therefore parallel to the horizontal orientation rod 212. The first Fox plane frame 250, which is coupled to the vertical orientation rod 210 proximate the horizontal orientation rod 212, can be oriented in a horizontal reference plane, which can be facilitated by using a detachable level 253 (see FIG. 10B) (e.g., a bubble level). The pupillary rod 252 and the first Fox plane frame 250 can facilitate anteriorly recording an occlusal plane of the patient, which may be parallel to the interpupillary line in most patients. The second Fox plane frame 251 can be positioned in a plane that includes or is parallel to the ala-tragus line (Camper's line), which runs from the interior border of the ala of the nose to the superior border of the tragus of the ear. The first Fox plane frame 250 and the second Fox plane frame 251 can facilitate posteriorly recording the occlusal plane of the patient, which may be parallel to the ala-tragus line in most patients. As most individuals vary from having perfectly perpendicular and perfectly proportioned features (for example the lips may be slightly crooked on the patient's face), the recordation of facial and dental features by the present technology accommodates variance in patient symmetry and anatomy and allows for the creation of dental prosthetics that appear more attractive on the patient.

In some embodiments, the vertical and horizontal orientation rods 210, 212 are fixed relative to one another (e.g., in orthogonal planes). In other embodiments, the vertical and horizontal orientation rods 210, 212 are rotatable relative to one another, such as to align with a sagittal midline reference plane and horizontal reference plane that are not orthogonal. In addition, the bite plate 220 can be movable (e.g., in one or more translational and/or rotational degrees of freedom) relative to the vertical and horizontal orientation rods 210, 212.

Once properly positioned, the orientation rods 210, 212, the connection system (e.g., including the nose extension arm 230), and the bite plate 220 may then be locked in place with respect to one another in order to record the desired planes. These components may be locked into place concurrently or independently. For example, a set screw or other mechanical fastener can be engaged. Optionally, a resin or glue can be hardened about the connection system joints to prevent movement subsequent to recording. A bite impression taken while the orientation planes are recorded may later be properly oriented, relative to the planes of the patient's head, by referring to the record made by the dental measurement device.

The vertical orientation rod 210, the horizontal orientation rod 212, connection system (e.g., including the nose extension arm 230), and the bite plate 220 containing an imprinted dental record of a patient can be provided to a dental prosthetic manufacturer such that the record of the planes may be used in the creation of the dental prosthetics. With the nose extension arm 230 and bite plate 220 detached from the orientation rods 210, 212, these components will fit in a smaller mailing package and can be reassembled upon arrival at the dental laboratory. Furthermore, removal of the orientation rods 210, 212 before shipment may help eliminate bending and damage during transport while ensuring that the dental lab receives an accurate bite plane orientation.

FIG. 14 illustrates a dental prosthetic manufacturing system 101 for use with an articulator 160 in accordance with an example of the present disclosure. The dental prosthetic manufacturing system 101 can utilize certain components and elements discussed above with regard to the dental measurement device 100 of FIGS. 1A-1D. For example, the system 101 can include the bite plate 120 containing an imprinted dental record 127 of the patient, and the nose extension arm 130 coupled to the bite plate 120 with the joint 131 set at a fixed position to record the position and orientation of the bite plate 120 relative to the patient's “baseline” features or landmarks. These components can be assembled at the laboratory with a vertical orientation rod 110, a baseline orientation frame 111, and (optionally) a horizontal orientation rod 112 to recreate the dental measurement device 100, which provides a way to mount the maxillary imprint 127 or cast on the bite plate 120 in the correct position and orientation on the articulator 160.

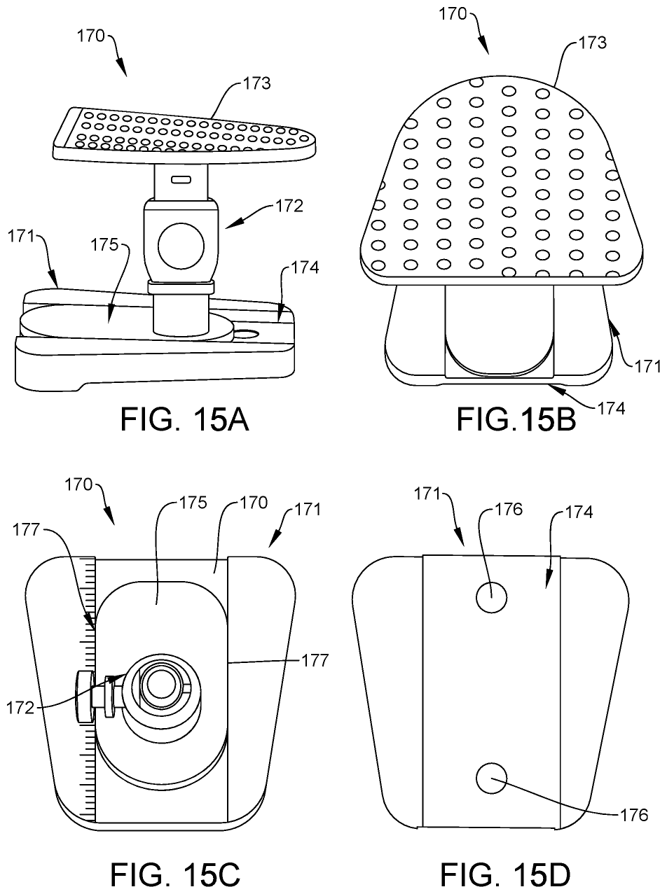

The dental prosthetic manufacturing system 101 can also include a stand or mounting table 170 (shown isolated in FIGS. 15A-15D) configured to couple with the articulator 160 and support the bite plate 120. The stand 170 can have a base 171 configured to interface with and couple to the articulator 160, and a body 172 extending from the base 171 to support the bite plate 120. In some embodiments, as shown in FIGS. 15A and 15B, the stand 170 can include a transfer plate 173 mounted to a top of the body 172. The transfer plate 173 can be configured to move vertically relative to the body 172. In some embodiments, the transfer plate 173 can be configured to rotate about a vertical axis relative to the body 172. In the illustrated embodiment, the body 172 can be configured to move or translate relative to the base 171. For example, the base 171 can include a recess or channel 174, and the body 172 can have a bottom portion 175 configured to fit within the channel 174 and slide along the channel 174 to change a position body 172 relative to the base 171. The base 171 can include magnets 176 in the channel 174 to maintain the bottom portion 175 in the channel 174. Interfacing sidewalls of the channel 174 and the bottom portion 175 can include teeth 177 to prevent unwanted movement of the body 172 relative to the base 171 along the channel 174.

In use, the stand 170 can be coupled to the articulator 160. The vertical orientation rod 110 can be coupled to the articulator 160 with a slider mount 161. An alternative slider mount 161′ is shown in FIG. 16 and has side attachment notches 162′ that are configured to interface with features of a given articulator to couple the slider mount 161′ to the articulator. The baseline orientation frame 111 can be coupled to the vertical orientation rod 110 in the same relative vertical location as when the dental imprint 127 was recorded, using the reference markings 118b as a guide. The bite plate 120 can be coupled to the nose extension arm 130, which can also be coupled to the vertical orientation rod 110. The slider mount 161 can be positioned horizontally relative to the condylar 163 and can enable vertical movement of the vertical orientation rod 110 to properly position the baseline orientation frame 111 relative to the condylar 163 of the articulator 160 using the reference markings 118a as a guide, which will properly locate the bite plane 120 relative to the condylar 163 to replicate the position of the maxillary arch relative to the patient's temporal mandibular joint. If the stand 170 includes the transfer plate 173, the bite plate 120 (i.e., the occlusal plate 122) can be coupled to the transfer plate 173, such as with a plaster or a dental stone. The body 172 can be positioned relative to the occlusal plate 122 to enable proper coupling of the occlusal plate 122 with the transfer plate 173 by sliding the body 172 along the channel 174. The direction of this sliding movement will typically be toward or away from the vertical orientation rod 110. For example, the baseline orientation frame 111 and the vertical orientation rod 110 define a vertical plane. The body 172 can be translatable relative to the base 171 in a direction parallel to the vertical plane. Once the plaster or dental stone has set, a model of the patient's maxillary arch can be properly positioned and oriented using the dental imprint 127 on the occlusal plate 122.

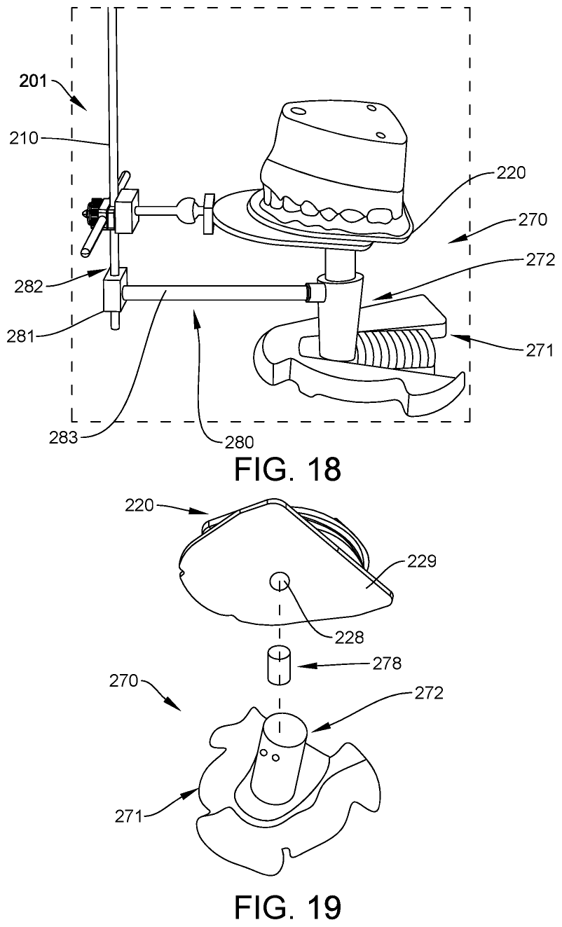

FIG. 17 illustrates a dental prosthetic manufacturing system 201 for use with an articulator 260 in accordance with another example of the present disclosure. The dental prosthetic manufacturing system 201 can utilize certain components and elements discussed above with regard to the dental measurement device 200 of FIGS. 9A and 9B. The dental measurement device 200 provided to the lab can be reassembled, if necessary, to include a vertical orientation rod 210, a horizontal orientation rod 212 fixedly coupled to the vertical orientation rod 210, and a bite plate 220 fixedly coupled to the vertical and horizontal orientation rods 210, 212. The bite plate 220 contains an imprinted dental record 227 of a patient. This dental measurement device 200 can be mounted on a stand 270 or set-up table for use in the articulator 260 (see also FIGS. 18 and 19) to construct a dental prosthetic. The stand 270 can have a base 271 configured to interface with and couple to the articulator 260, and a body 272 extending from the base 271. The bite plate 220 can have a ball 228 or a socket on a bottom side of the bite plate 220, and the stand 270 can have the other of the ball or the socket 278 coupled to a top of the body 272 to facilitate coupling with the bite plate 220. In one aspect, as shown in FIG. 19, the bite plate 220 can be mounted on a platform 229 that includes the ball 228 or a socket for coupling the bite plate 220 to the stand 270. The ball and socket coupling mechanism can provide multiple rotational degrees of freedom to accommodate multiple degrees of freedom in which the bite plate 220 may be oriented relative to the vertical and horizontal orientation rods 210, 212.

In one alternative, a level can be removably associated with one or more of the stand base or occlusal table. The additional level can further improve correlation of position information between the system and a mold, especially used in connection with levels as previously described (e.g. Fox plane frame, interpupillary bars, nose extension member, etc.).

In some embodiments, as shown in FIG. 18, the system 201 can include a lateral support arm 280 coupleable to the vertical orientation rod 210 and the body 272 of the stand 270, which can provide stability for the dental measurement device 200 and facilitate proper orientation of the vertical and horizontal orientation rods 210, 212 relative to the stand 270. In one aspect, the lateral support arm 280 can be magnetically coupleable to the body 272. For example, a magnetic coupler can include a mating protrusion and recess, each containing magnets or a magnet and a magnetic material. The protrusion and recess can include a shape or “key” feature that can establish and maintain a relative orientation of the components to hold the lateral support arm in a desired orientation relative to the stand (e.g., with the vertical orientation arm oriented vertically).

In another aspect, the lateral support arm 280 can be configured to facilitate vertical movement and/or horizontal movement of the vertical orientation rod 210 relative to the body 272. For example, an attachment fitting 281 of the lateral support arm 280 can have an attachment opening 282 configured to facilitate upward or downward sliding of the vertical orientation rod 210 under manually applied force but maintain a fixed relationship at rest when only subject to forces due to gravity. In addition, the lateral support arm 280 can include one or more rails 283 configured to facilitate a sliding engagement with the attachment fitting 281 toward or away from the stand 270.

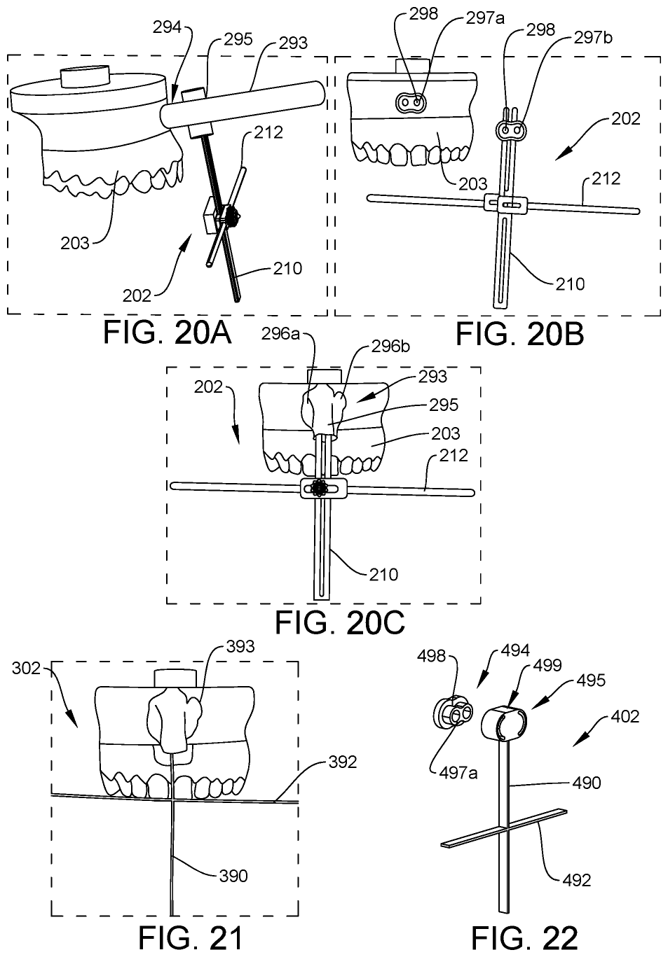

FIGS. 20A-22 illustrate reference tools for modeling a dental prosthetic. In general, a reference tool can include a vertical reference bar, a horizontal reference bar, and a magnetic coupler for magnetically coupling the reference tool to a dental prosthetic. In one embodiment of a reference tool 202, shown in FIGS. 20A-20C, the vertical and horizontal reference bars can be the vertical and horizontal orientation rods 210, 212 of the dental measurement device 200. In other embodiments of reference tools 302, 402, shown in FIGS. 21 and 22, respectively, the vertical and horizontal reference bars 390, 392 and 490, 492 can be integrally formed in a monolithic structure. Typically, the vertical and horizontal reference bars 390, 392 and 490, 492 of the respective reference tools 302, 402 will be fixedly coupled at 90 degrees to one another, which is useful when the sagittal midline reference plane and horizontal reference plane that are orthogonal.

The reference tool 202 can include a lateral offset arm 293 (see FIG. 20A) coupled between the vertical reference bar 210 and a magnetic coupler 294. The reference tool 302 includes a similar lateral offset arm 393. In one aspect, the lateral offset arm 293 can be configured to facilitate at least one of vertical movement or horizontal movement of the vertical reference bar 210 relative to a dental prosthetic 203. For example, an attachment fitting 295 of the lateral offset arm 293 can have an attachment opening configured to facilitate upward or downward sliding of the vertical bar 210 under manually applied force but maintain a fixed relationship at rest when only subject to forces due to gravity. In addition, the lateral offset arm 293 can include one or more rails 296a, 296b configured to facilitate a sliding engagement with the attachment fitting 295 toward or away from the dental prosthetic 203. In one aspect, the magnetic coupler 294 can be rotatable relative to the rails 296a, 296b. The reference tool 402 is without a lateral offset arm.

The magnetic coupler 294 can include a mating protrusion 297a and recess 297b, each containing magnets 298 or a magnet and a magnetic material. The protrusion 297a and recess 296 can include a shape or “key” feature that can establish and maintain a relative orientation of the components to hold the reference tool 202 in a desired orientation relative to the dental prosthetic. The reference tool 402 includes a similar magnetic coupler 494, which includes mating protrusion 495 and recess (hidden from view) and magnets 497 or a magnet and a magnetic material. The protrusion and recess include a shape or “key” feature that can establish and maintain a relative orientation of the components to hold the reference tool 402 in a desired orientation relative to the dental prosthetic.

The reference tool 402 can also include an attachment fitting 495 with an attachment opening 499 configured to facilitate upward or downward sliding of the vertical bar 490 under manually applied force but maintain a fixed relationship at rest when only subject to forces due to gravity.

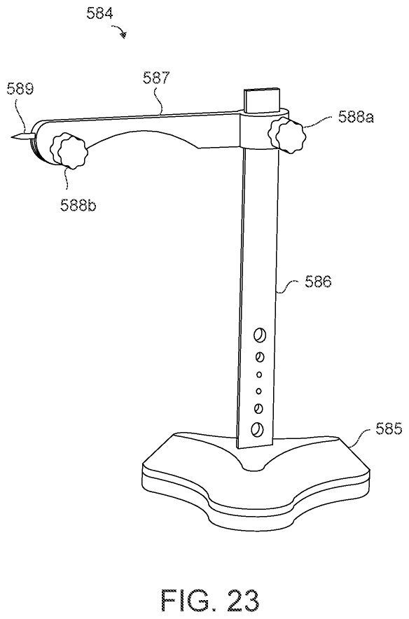

FIG. 23 illustrates a stylus 584 that can be used by a dental technician or a dentist to co-relate a horizontal position of a baseline and an extension arm (e.g., the extension arm 130, 130′ discussed herein) in a horizontal line marking (e.g., with pencil lead or graphite) on a stone maxillary model that will be mounted on an articulator. This can enable the stone model to be ground down parallel to the horizontal baseline to maintain the correct position of the maxillary arch. The stylus 584 can have a stylus base 585 operable to interface with, and be supported by, a support surface. In one aspect, the stylus base 585 can be configured to move freely relative to the support surface. For example, the stylus base 585 can include a wheel, a roller, a ball, etc. (e.g., three spherical metal balls) mounted on a bottom side of the stylus base 585 to allow the stylus to glide freely on a flat support surface. The stylus 584 can also have a vertical stylus arm 586 vertically extending from the stylus base 585, and a horizontal stylus arm 587 horizontally extending from the vertical stylus arm 586. The horizontal stylus arm 587 can be vertically movable relative to the vertical stylus arm 586 to adjust a vertical position of the horizontal stylus arm 587 to capture the correct baseline level. The horizontal stylus arm 587 can be secured to the vertical stylus arm 586 in any suitable manner, such as by a threaded fastener 588a (e.g., one or more of a threaded rod, a nut, a screw, etc.), a clamp, a cam mechanism, etc. In addition, the stylus 584 can include a marking instrument 589 (e.g., pencil lead or graphite) supported by the horizontal stylus arm 587. The marking instrument 589 can be removably attached to the horizontal stylus arm 587 for easy replacement when worn or depleted. The marking instrument 589 can be removably attached to the horizontal stylus arm 587 in any suitable manner, such as by a threaded fastener 588b (e.g., one or more of a threaded rod, a nut, a screw, etc.), a clamp, a cam mechanism, etc.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A scannable dental spatial orientation device, comprising:
a coupling feature operable to couple with a bite plate for supporting a physical dental record material;
a body portion having first, second, and third planar faces oriented orthogonal to one another; and
at least one joint configured for rotating the bite plate and the body portion relative to one another to align the first, second, and third planar faces parallel to a sagittal midline reference plane, a horizontal plane, and a frontal plane of a patient.

2. The device of claim 1, wherein the at least one joint is configured for rotating the bite plate relative to the body portion in a direction parallel to at least one of the first, second, or third planar faces.

3. The device of claim 1, wherein the at least one joint comprises a ball and socket joint.

4. The device of claim 1, wherein at least one of the first, second, or third planar faces comprises an optical sensor recognition feature configured to facilitate recognition of the at least one of the first, second, or third planar faces by an optical sensor.

5. The device of claim 4, wherein the optical sensor recognition feature comprises at least one of a matte finish, a surface irregularity, a texture, a shape, a pattern, an alphanumeric character, or indicia.

6. The device of claim 5, wherein the pattern comprises at least one of a reticulate pattern, a parametric pattern, or a fractal pattern.

7. The device of claim 5, wherein the shape comprises at least one of a geometric shape or a freeform shape.

8. The device of claim 5, wherein the indicia comprises at least one of a company name, a logo, or a trademark.

9. The device of claim 4, wherein the optical sensor recognition feature comprises at least one of a two-dimensional feature or a three-dimensional feature.

10. The device of claim 1, wherein the body portion comprises a rectangular cuboid configuration.

11. The device of claim 1, further comprising a second coupling feature operable to couple with a vertical orientation rod to facilitate aligning the first, second, and third planar faces parallel to the sagittal midline reference plane, the horizontal plane, and the frontal plane of the patient.

12. A dental record optical scan system, comprising:
a physical dental record of a maxillary arch of a patient;
a scannable dental spatial orientation device associated with the physical dental record, the scannable dental spatial orientation device including a body portion having first, second, and third planar faces oriented orthogonal to one another and representing planes aligned parallel to a sagittal midline reference plane, a horizontal plane, and a frontal plane of the patient relative to the physical dental record;
a support structure operable to support the physical dental record and the scannable dental spatial orientation device for optical scanning; and
an optical scanner operable to scan the physical dental record and the scannable dental spatial orientation device with an optical sensor to create a digital dental record and a digital representation of the first, second, and third planar faces, wherein the digital dental record is established for a position of the maxillary arch relative to the sagittal midline reference plane, the horizontal plane, and the frontal plane of the patient.

13. The system of claim 12, wherein at least one of the first, second, or third planar faces comprises an optical sensor recognition feature configured to facilitate recognition of the at least one of the first, second, or third planar faces by the optical sensor.

14. The system of claim 12, wherein the optical scanner comprises at least one of an intraoral scanner or a desktop dental scanner.

15. The system of claim 12, wherein the support structure comprises a base portion and a support arm extending vertically from the base portion, the support arm being configured to interface and couple with the scannable dental spatial orientation device, and the base portion being operable to support the physical dental record and the scannable dental spatial orientation device relative to a support surface for optical scanning.

16. The system of claim 16, wherein the support arm comprises at least one of a straight configuration or a curved configuration.

17. The system of claim 16, wherein the base portion comprises a level to facilitate orienting one of the first, second, or third planar faces in a horizontal plane.

18. The system of claim 12, wherein the physical dental record is supported by a bite plate comprising an optical sensor recognition feature configured to facilitate recognition of a planar face of the bite plate by the optical sensor.

19. A method for digitally recording a maxillary arch position, comprising:
obtaining a physical dental record of a maxillary arch of a patient and a scannable dental spatial orientation device associated with the physical dental record, the scannable dental spatial orientation device including a body portion having first, second, and third planar faces oriented orthogonal to one another and representing planes aligned parallel to a sagittal midline reference plane, a horizontal plane, and a frontal plane of the patient relative to the physical dental record;
coupling the scannable dental spatial orientation device to a support structure operable to support the physical dental record and the scannable dental spatial orientation device for optical scanning; and
optically scanning the physical dental record and the scannable dental spatial orientation device to create a digital dental record and a digital representation of the first, second, and third planar faces, wherein the digital dental record is established for a position of the maxillary arch relative to the sagittal midline reference plane, the horizontal plane, and the frontal plane of the patient.

20. The method of claim 19, wherein optically scanning the physical dental record and the scannable dental spatial orientation device comprises scanning the physical dental record and the scannable dental spatial orientation device with at least one of an intraoral scanner or a desktop dental scanner.

21. The method of claim 19, wherein at least one of the first, second, or third planar faces comprises an optical sensor recognition feature configured to facilitate recognition of the at least one of the first, second, or third planar faces by an optical sensor.

22. The method of claim 19, wherein coupling the scannable dental spatial orientation device to a support structure comprises attaching the scannable dental spatial orientation device to the support structure with a threaded fastener.
